(19) United States
(12) Patent Application Publication
(10) Pub. No.: US 2016/0152530 A1
(43) Pub. Date: Jun. 2, 2016

(54) METHOD FOR THE OXIDATIVE DEHYDROGENATION OF N-BUTENES TO BUTADIENE

(71) Applicant: BASF SE, Ludwigshafen (DE)

(72) Inventors: Philipp GRÜNE, Mannheim (DE); Wolfgang RÜTTINGER, Mannheim (DE); Christine SCHMITT, Mannheim (DE); Christian WALSDORFF, Ludwigshafen (DE)

(21) Appl. No.: 14/903,813

(22) PCT Filed: Jul. 7, 2014
(86) PCT No.: PCT/EP2014/064409

§ 371 Date: Jan. 8, 2016

(30) Foreign Application Priority Data
Jul. 10, 2013 (EP) 13175935.9

(51) Int. Cl. C07C 5/48

(52) U.S. Cl. CPC C07C 5/48; C07C 2523/745; C07C 2523/75; C07C 2523/31; C07C 2523/28; C07C 2523/26; C07C 2523/04

(57) ABSTRACT

The invention relates to a process for the oxidative dehydrogenation of n-butenes to butadiene, which comprises two or more production steps (i) and at least one regeneration step (ii) and in which
(i) a starting gas mixture comprising n-butenes is mixed with an oxygen-comprising gas in a production step and the mixed gas is brought into contact with a multimetal oxide catalyst which comprises at least molybdenum and a further metal and is arranged in a fixed catalyst bed at a temperature of from 220 to 490° C. in a fixed-bed reactor, with a product gas mixture comprising at least butadiene, oxygen and water vapor being obtained at the outlet of the fixed-bed reactor,
   and
(ii) the multimetal oxide catalyst is regenerated in a regeneration step by passing an oxygen-comprising regeneration gas mixture over the fixed catalyst bed at a temperature of from 200 to 450° C. and burning off the carbon deposited on the catalyst,
   with a regeneration step (ii) being carried out between two production steps (i),
   wherein the oxygen content in the product gas mixture at the outlet of the fixed-bed reactor is at least 5% by volume and the duration of a production step (i) is not more than 1000 hours.

The invention relates to a process for the oxidative dehydrogenation of n-butenes to butadiene.

Butadiene is an important basic chemical and is used, for example, for the production of synthetic rubbers (butadiene homopolymers, styrene-butadiene rubber or nitrile rubber) or for the preparation of thermoplastic terpolymers (acrylonitrile-butadiene-styrene copolymers). Butadiene is also converted into sulfolane, chloroprene and 1,4-hexamethylenediamine (via 1,4-dichlorobutene and adiponitrile). Furthermore, vinylcyclohexene can be produced by dimerization of butadiene and this vinylcyclohexene can be dehydrogenated to styrene.

Butadiene can be prepared by thermal cracking (steam cracking) of saturated hydrocarbons, usually employing naphtha as raw material. Steam cracking of naphtha gives a hydrocarbon mixture composed of methane, ethane, ethene, acetylene, propane, propene, propyne, allene, butanes, butenes, butadiene, butynes, methylallene, C₅-hydrocarbons and higher hydrocarbons.

Butadiene can also be obtained by oxidative dehydrogenation of n-butenes (1-butene and/or 2-butene). As starting gas mixture for the oxidative dehydrogenation of n-butenes to butadiene, it is possible to use any mixture comprising n-butenes. For example, it is possible to use a fraction which comprises n-butenes (1-butene and/or 2-butene) as main constituent and has been obtained from the C₄ fraction from a naphtha cracker after separating off butadiene and isobutene. Furthermore, gas mixtures which comprise 1-butene, cis-2-butene, trans-2-butene or mixtures thereof and have been obtained by dimerization of ethylene can also be used as starting gas. In addition, gas mixtures which comprise n-butenes and have been obtained by fluid catalytic cracking (FCC) can also be used as starting gas.

Gas mixtures which comprise n-butenes and can be used as starting gas in the oxidative dehydrogenation of n-butenes to butadiene can also be produced by nonoxidative dehydrogenation of n-butane-comprising gas mixtures.

WO2009/124945 discloses a coated catalyst for the oxidative dehydrogenation of 1-butene and/or 2-butene to butadiene, which can be obtained from a catalyst precursor comprising
(a) a support body,
(b) a shell comprising (i) a catalytically active multimetal oxide which comprises molybdenum and at least one further metal and has the general formula

Mo₁₂BiₐCr_{b}X¹_{c}Fe_{d}X²ₑX³_{f}O_{y}

where
X¹=Co and/or Ni,
X²=Si and/or AI,
X³=Li, Na, K, Cs and/or Rb,
0.2≦a≦1,
0≦b≦2,
2≦c≦10,
0.55≦d≦10,
0≦e≦10,
0≦f≦0.5 and
y=a number which is determined by the valence and abundance of the elements other than oxygen so as to result in charge neutrality.

WO 2010/137595 discloses a multimetal oxide catalyst for the oxidative dehydrogenation of alkenes to dienes, which comprises at least molybdenum, bismuth and cobalt and has the general formula

MoₐBi_{b}Co_{c}Ni_{d}FeₑX_{f}Y_{g}ZₕSiᵢOⱼ.

In this formula, X is at least one element from the group consisting of magnesium (Mg), calcium (Ca), zinc (Zn), cerium (Ce) and samarium (Sm). Y is at least one element from the group consisting of sodium (Na), potassium (K), rubidium (Rb), cesium (Cs) and thallium (Tl). Z is at least one element from the group consisting of boron (B), phosphorus (P), arsenic (As) and tungsten (W), a-j represent the atom fraction of the respective element, where a=12, b=0.5-7, c=0-10, d=0-10, (where c+d=1-10), e=0.05-3, f=0-2, g=0.04-2, h=0-3 and i=5-48. In the examples, a catalyst having the composition Mo₁₂Bi₅Co_{2.5}Ni_{2.5}Fe_{0.4}Na_{0.35}B_{0.2}K_{0.08}Si₂₄ is used in the form of pellets having a diameter of 5 mm and a height of 4 mm in the oxidative dehydrogenation of n-butenes to butadiene.

In the oxidative dehydrogenation of n-butenes to butadiene, precursors of carbonaceous material, for example styrene, anthraquinone and fluorenone can be formed and ultimately lead to carbonization and deactivation of the multimetal oxide catalyst. The pressure drop over the catalyst bed can increase as a result of formation of carbon-comprising deposits. It is possible to regenerate the catalyst by burning off carbon deposited on the multimetal oxide catalyst at regular intervals by means of an oxygen-comprising gas in order to restore the activity of the catalyst.

JP 60-058928 describes the regeneration of a multimetal oxide catalyst for the oxidative dehydrogenation of n-butenes to butadiene, which comprises at least molybdenum, bismuth, iron, cobalt and antimony using an oxygen-comprising gas mixture at a temperature of from 300 to 700° C., preferably from 350 to 650° C., and an oxygen concentration of from 0.1 to 5% by volume. As oxygen-comprising gas mixture, air diluted with suitable inert gases such as nitrogen, steam or carbon dioxide is fed in.

WO 2005/047226 describes the regeneration of a multimetal oxide catalyst for the partial oxidation of acrolein to acrylic acid, which comprises at least molybdenum and vanadium, by passing an oxygen-comprising gas mixture over the catalyst at a temperature of from 200 to 450° C. Preference is given to using lean air comprising from 3 to 10% by volume of oxygen as oxygen-comprising gas mixture. Apart from oxygen and nitrogen, the gas mixture can comprise water vapor.

JP 2012077074 describes the breaking up of the catalyst by excessive carbonization. The high degree of carbonization is said to be controlled by means of a suitable choice of the concentration of oxygen and hydrocarbons (in particular butenes) in the feed gas mixture.

One problem is to determine the point in time at which regeneration of the catalyst is to be carried out. For example, the oxydehydrogenation can be carried out until the drop in activity of the catalyst has reached a particular prescribed value, or else the pressure drop over the reactor has reached a particular prescribed value. However, at this point in time carbonization of the catalyst has already progressed to a great extent. However, advanced formation of carbonaceous material on the catalyst surface and within the catalyst can reduce the mechanical stability of the catalyst, which can lead to flaking off of active composition and damage to the catalyst. Active composition which has flaked off can collect in an uncontrolled manner in the reaction tubes of the tube or shell-and-tube reactor or outside these. Reliable operation of the plant may then no longer be possible.

It is an object of the invention to provide a process for the oxidative dehydrogenation of n-butenes to butadiene in a fixed-bed reactor, in which process damage to the catalyst during operation of the fixed-bed reactor is minimized.

This object is achieved by a process for the oxidative dehydrogenation of n-butenes to butadiene, which comprises two or more production steps (i) and at least one regeneration step (ii) and in which

(i) a starting gas mixture comprising n-butenes is mixed with an oxygen-comprising gas in a production step and the mixed gas is brought into contact with a multimetal oxide catalyst which comprises at least molybdenum and a further metal and is arranged in a fixed catalyst bed at a temperature of from 220 to 490° C. in a fixed-bed reactor, with a product gas mixture comprising at least butadiene, oxygen and water vapor being obtained at the outlet of the fixed-bed reactor,

and

(ii) the multimetal oxide catalyst is regenerated in a regeneration step by passing an oxygen-comprising regeneration gas mixture over the fixed catalyst bed at a temperature of from 200 to 450° C. and burning off the carbon deposited on the catalyst,

with a regeneration step (ii) being carried out between two production steps (i),

wherein the oxygen content in the product gas mixture at the outlet of the fixed-bed reactor is at least 5% by volume and the duration of a production step (i) is not more than 1000 hours.

It has surprisingly been found that despite an oxygen content in the product gas mixture at the outlet of the oxydehydrogenation reactor of at least 5% by volume, carbonization of the catalyst occurs over the long term. This carbonization of the catalyst initially does not become apparent in a drop in activity or in a decrease in selectivity. In general at the point in time when the regeneration step (ii) is carried out, the conversion of the n-butenes has decreased by not more than 2% during the preceding 200 hours of the production step (i). The regeneration step (ii) is thus generally carried out when the conversion of the n-butenes has decreased by not more than 2% in the last 200 hours of the production step (i).

Rapid carbonization can be prevented by means of an oxygen content of at least 5% by volume in the product gas mixture. Delimiting a production step (i) to 1000 hours prevents damage to the catalyst caused by long-term carbonization.

In general, a production step (i) has a duration of not more than 1000 hours, preferably not more than 670 hours, in particular preferably not more than 340 hours. In general, a production step (i) has a length of 20 hours, preferably at least 90 hours and particularly preferably at least 160 hours. The catalyst can go through up to 5000 or more cycles of production and regeneration steps.

Catalysts suitable for the oxydehydrogenation are generally based on an Mo—Bi—O-comprising multimetal oxide system which generally additionally comprises iron. In general, the catalyst system further comprises additional components from group 1 to 15 of the Periodic Table, for example potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

In general, the catalytically active multimetal oxide comprising molybdenum and at least one further metal has the general formula (I)

Mo₁₂BiₐFe_{b}CO_{c}Ni_{d}CrₑX¹_{f}X²_{g}Oₓ (I),

the variables having the following meanings:

X¹=W, Sn, Mn, La, Ce, Ge, Ti, Zr, Hf, Nb, P, Si, Sb, Al, Cd and/or Mg;

X²=Li, Na, K, Cs and/or Rb,

a=0.1 to 7, preferably from 0.3 to 1.5;

b=0 to 5, preferably from 2 to 4;

c=0 to 10, preferably from 3 to 10;

d=0 to 10;

e=0 to 5, preferably from 0.1 to 2;

f=0 to 24, preferably from 0.1 to 2;

g=0 to 2, preferably from 0.01 to 1; and

x=a number determined by the valence and abundance of the elements other than oxygen in (I).

The catalyst can be an all-active catalyst or a coated catalyst. If it is a coated catalyst, it comprises a support body (a) and a shell (b) comprising the catalytically active multimetal oxide comprising molybdenum and at least one further metal.

Support materials suitable for coated catalysts are, for example, porous or preferably nonporous aluminum oxides, silicon dioxide, zirconium dioxide, silicon carbide or silicates such as magnesium silicate or aluminum silicate (e.g. steatite of the type C 220 from CeramTec). Materials of the support body are chemically inert.

The support materials can be porous or nonporous. The support material is preferably nonporous (total volume of the pores based on the volume of the support body preferably ≦1% by volume).

The use of essentially nonporous, spherical steatite supports (e.g. steatite of the type C 220 from CeramTec) which have a rough surface and a diameter of from 1 to 8 mm, preferably from 2 to 6 mm, particularly preferably from 2 to 3 or from 4 to 5 mm, is particularly suitable. However, the use of cylinders of support material having a length from 2 to 10 mm and an external diameter of from 4 to 10 mm as support bodies is also appropriate. In the case of rings as support bodies, the wall thickness is usually from 1 to 4 mm. Preferred ring-shaped support bodies have a length of from 2 to 6 mm, an external diameter of from 4 to 8 mm and a wall thickness of from 1 to 2 mm. Rings having a geometry of 7 mm×3 mm×4 mm (external diameter×length×internal diameter) are also particularly useful as support bodies. The layer thickness of the shell (b) composed of a multimetal oxide composition comprising molybdenum and at least one further metal is generally from 5 to 1000 μm. Preference is given to from 10 to 800 μm, particularly preferably from 50 to 600 μm and very particularly preferably from 80 to 500 μm.

Examples of Mo—Bi—Fe—O-comprising multimetal oxides are Mo—Bi—Fe—Cr—O— or Mo—Bi—Fe—Zr—O-comprising multimetal oxides. Preferred systems are, for example, described in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo₁₂BiFe₃Co_{4.5}Ni_{2.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFeCo_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and their production are also described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia)

where

X¹=Si, Mn and/or Al,

X²=Li, Na, K, Cs and/or Rb,

0.2≦a≦1,

0.5≦b≦10,

0≦c≦10,

0≦d≦10,

2≦c+d≦10

0≦e≦2,

0≦f≦10

0≦g≦0.5

y=a number determined by the valence and abundance of the elements other than oxygen in (Ia) so as to result in charge neutrality.

Preference is given to catalysts whose catalytically active oxide composition has only Co, of the two metals Co and Ni (d=0). X¹ is preferably Si and/or Mn and X² is preferably K, Na and/or Cs, with particular preference being given to X²=K.

The stoichiometric coefficient a in formula (Ia) is preferably 0.4≦a≦1, particularly preferably 0.4≦a≦0.95. The value for the variable b is preferably in the range 1≦b≦5 and particularly preferably in the range 2≦b≦4. The sum of the stoichiometric coefficients c+d is preferably in the range 4≦c+d≦8 and particularly preferably in the range 6≦c+d≦8. The stoichiometric coefficient e is preferably in the range 0.1≦e≦2 and particularly preferably in the range 0.2≦e≦1. The stoichiometric coefficient g is advantageously ≧0. Preference is given to 0.01≦g≦0.5 and particularly preferably 0.05≦g≦0.2.

The value of the stoichiometric coefficient of oxygen, y, is determined by the valence and abundance of the cations so as to result in charge neutrality. Coated catalysts according to the invention having catalytically active oxide compositions whose molar ratio of Co/Ni is at least 2:1, preferably at least 3:1 and particularly preferably 4:1, are useful. It is best for only Co to be present.

The coated catalyst is produced by applying a layer comprising the multimetal oxide comprising molybdenum and at least one further metal to the support body by means of a binder, and drying and calcining the coated support body.

Finely divided multimetal oxides comprising molybdenum and at least one further metal which are to be used according to the invention can be obtained essentially by producing an intimate dry mixture from starting compounds of the elemental constituents of the catalytically active oxide composition and thermally treating the intimate dry mixture at a temperature of from 150 to 650° C.

Production of the Multimetal Oxide Catalyst To produce suitable finely divided multimetal oxide compositions, known starting compounds of the elemental constituents other than oxygen of the desired multimetal oxide composition are used as starting materials in the respective stoichiometric ratio and a very intimate, preferably finely divided, dry mixture is produced from these and is then subject to the thermal treatment.

Here, the sources can be either oxides or compounds which can be converted into the oxides by heating, at least in the presence of oxygen. Possible starting compounds apart from the oxides are therefore, in particular, halides, nitrates, formates, oxalates, acetates, carbonates or hydroxides.

Suitable starting compounds of molybdenum also include its oxy compounds (molybdates) or the acids derived therefrom.

Suitable starting compounds of Bi, Cr, Fe and Co are, in particular, the nitrates thereof.

The intimate mixing of the starting compounds can in principle be carried out in dry form or in the form of the aqueous solutions or suspensions.

An aqueous suspension can be produced, for example, by combining a solution comprising at least molybdenum and an aqueous solution comprising the other metals. Alkali metals or alkaline earth metals can be present in both solutions. Combining the solutions results in a precipitation to form a suspension. The temperature of the precipitation can be above room temperature, preferably from 30° C. to 95° C. and particularly preferably from 35° C. to 80° C. The suspension can then be aged for a particular period of time at elevated temperature. The aging time is generally in the range from 0 to 24 hours, preferably from 0 to 12 hours and particularly preferably from 0 to 8 hours. The temperature of aging is generally in the range from 20° C. to 99° C., preferably from 30° C. to 90° C. and particularly preferably from 35° C. to 80° C. During precipitation and aging of the suspension, the latter is generally mixed by stirring. The pH of the mixed solutions or suspension is generally in the range from pH 1 to pH 12, preferably from pH 2 to pH 11 and particularly preferably from pH 3 to pH 10.

Removal of the water gives a solid which is an intimate mixture of the metal components introduced. The drying step can in general be carried out by evaporation, spray drying or freeze drying or the like. Drying is preferably carried out by spray drying. For this purpose, the suspension is atomized at elevated temperature by means of a spray head whose temperature is generally from 120° C. to 300° C. and the dried product is collected at a temperature of >60° C. The residual moisture, determined by drying of the spray powder at 120° C., is generally less than 20% by weight, preferably less than 15% by weight and particularly preferably less than 12% by weight.

To produce all-active catalysts, the spray-dried powder is converted into a shaped body in a further step. As shaping aids (lubricants), it is possible to use, for example, water, boron trifluoride or graphite. Based on the composition to be shaped to form the shaped catalyst precursor body, use is generally made of ≦10% by weight, usually ≦6% by weight, often ≦4% by weight, of shaping aid. The abovementioned amount added is usually >0.5% by weight. A preferred lubricant is graphite.

The thermal treatment of the shaped catalyst precursor body is generally carried out at temperatures above 350° C. However, a temperature of 650° C. is normally not exceeded in the thermal treatment. According to the invention, it is advantageous for a temperature of 600° C., preferably a temperature of 550° C. and particularly preferably a temperature of 500° C., not to be exceeded in the thermal treatment. Furthermore, a temperature of 380° C., advantageously a temperature of 400° C., especially advantageously a temperature of 420° C. and very particularly preferably a temperature of 440° C., is preferably exceeded in the thermal treatment of the shaped catalyst precursor body in the process of the invention. The thermal treatment can be divided into a plurality of segments over time. For example, a thermal treatment at a temperature of from 150 to 350° C., preferably from 220 to 280° C., can firstly be carried out, followed by a thermal treatment at a temperature of from 400 to 600° C., preferably from 430 to 550° C. The thermal treatment of the shaped catalyst precursor body normally takes a number of hours (usually more than 5 hours). The total duration of the thermal treatment frequently extends to more than 10 hours. Treatment times of 45 hours or 35 hours are usually not exceeded in the thermal treatment of the shaped catalyst precursor body. The total treatment time is often below 30 hours. Preference is given to 500° C. not being exceeded in the thermal treatment of the shaped catalyst precursor body and the treatment time in the temperature window of ≧400° C. extending to from 5 to 30 hours.

The thermal treatment (calcination) of the shaped catalyst precursor bodies can be carried out either under inert gas or under an oxidative atmosphere, e.g. air, and also under a reducing atmosphere (e.g. in mixtures of inert gas, NH₃, CO and/or H₂ or methane). It goes without saying that the thermal treatment can also be carried out under reduced pressure. The thermal treatment of the shaped catalyst precursor bodies can in principle be carried out in a wide variety of furnace types, e.g. heatable convection chambers, tray furnaces, rotary tube furnaces, belt calciners or shaft furnaces. The thermal treatment of the shaped catalyst precursor bodies is preferably carried out in a belt calcination apparatus as recommended by DE-A 10046957 and WO 02/24620. The thermal treatment of the shaped catalyst precursor bodies below 350° C. generally follows the thermal decomposition of the sources of the elemental constituents of the desired catalyst present in the shaped catalyst precursor bodies. This decomposition phase frequently occurs during heating to temperatures of <350° C. in the process of the invention.

To produce a coated catalyst, the catalytically active metal oxide composition obtained after the calcination can subsequently be converted by milling into a finely divided powder which is then applied with the aid of a liquid binder to the outer surface of a support body. The fineness of the catalytically active oxide composition applied to the surface of the support body is matched to the desired shell thickness.

Support materials suitable for producing coated catalysts are porous or preferably nonporous aluminum oxides, silicon dioxide, zirconium dioxide, silicon carbide or silicates such as magnesium silicate or aluminum silicate (e.g. steatite of the type C 220 from CeramTec). The materials of the support bodies are chemically inert.

The support materials can be porous or nonporous. The support material is preferably nonporous (total volume of the pores, based on the volume of the support body, preferably ≦1% by volume).

Preferred hollow cylinders as support bodies have a length of from 2 to 10 mm and an external diameter of from 4 to 10 mm. The wall thickness is preferably from 1 to 4 mm. Particularly preferred ring-shaped support bodies have a length of from 2 to 6 mm, an external diameter of from 4 to 8 mm and a wall thickness of from 1 to 2 mm. An example is provided by rings having the geometry 7 mm×3 mm×4 mm (external diameter×length×internal diameter) as support bodies.

The layer thickness D of a multimetal oxide composition comprising molybdenum and at least one further metal is generally from 5 to 1000 μm. Preference is given to from 10 to 800 μm, particularly preferably from 50 to 600 μm and very particularly preferably from 80 to 500 μm.

The application of the multimetal oxide comprising molybdenum and at least one further metal to the surface of the support body can be carried out according to the processes described in the prior art, for example as described in US-A 2006/0205978 and EP-A 0 714 700.

In general, the finely divided metal oxide compositions are applied to the surface of the support body or to the surface of the first layer with the aid of a liquid binder. Possible liquid binders are, for example, water, an organic solvent or a solution of an organic substance (e.g. an organic solvent) in water or in an organic solvent.

A solution consisting of from 20 to 95% by weight of water and from 5 to 80% by weight of an organic compound is particularly advantageously used as liquid binder. The organic proportion of the abovementioned liquid binders is preferably from 10 to 50% by weight and particularly preferably from 10 to 30% by weight.

Preference is generally given to organic binders or binder components whose boiling point or sublimation temperature at atmospheric pressure (1 atm) is ≧100° C., preferably ≧150° C. The boiling point or sublimation point of such organic binders or binder components at atmospheric pressure is very particularly preferably at the same time below the highest calcination temperature employed in the production of the finely divided multimetal oxide comprising molybdenum. This maximum calcination temperature is usually ≦600° C., frequently ≦500° C.

Examples of organic binders are monohydric or polyhydric organic alcohols, e.g. ethylene glycol, 1,4-butanediol, 1,6-hexanediol or glycerol, monobasic or polybasic organic carboxylic acids such as propionic acid, oxalic acid, malonic acid, glutaric acid or maleic acid, amino alcohols such as ethanolamine or diethanolamine and also monofunctional or polyfunctional organic amides such as formamide. Suitable organic binder promoters which are soluble in water, in an organic liquid or in a mixture of water and an organic liquid are, for example, monosaccharides and oligosaccharides such as glucose, fructose, sucrose and/or lactose.

Particularly preferred liquid binders are solutions consisting of from 20 to 95% by weight of water and from 5 to 80% by weight of glycerol. The glycerol content in these aqueous solutions is preferably from 5 to 50% by weight and particularly preferably from 8 to 35% by weight.

The application of the finely divided multimetal oxide comprising molybdenum can be carried out by dispersing the finely divided composition composed of multimetal oxide comprising molybdenum in the liquid binder and spraying the resulting suspension onto moving and optionally hot support bodies, as described in DE-A 1642921, DE-A 2106796 and DE-A 2626887. After spraying is complete, the moisture content of the resulting coated catalysts can, as described in DE-A 2909670, be reduced by passing hot air over the catalyst bodies.

Pore formers such as malonic acid, melamine, nonylphenol ethoxylate, stearic acid, glucose, starch, fumaric acid and succinic acid can additionally be added to the finely divided multimetal oxide with which the support is coated in order to produce a suitable pore structure of the catalyst and to improve the mass transfer properties. The catalyst preferably does not contain any pore formers.

However, preference is given to firstly moistening the support bodies with the liquid binder and subsequently applying the finely divided composition composed of multimetal oxide to the surface of the binder-moistened support body by rolling the moistened support bodies in the finely divided composition. To achieve the desired layer thickness, the above-described process is preferably repeated a number of times, i.e. the support body having the base coat is moistened again and then coated by contact with dry finely divided composition.

To carry out the process on the industrial scale, it is advisable to employ the process disclosed in DE-A 2909671, but preferably using the binders recommended in EP-A 714700. That is to say, the coated support bodies are introduced into a preferably inclined (angle of inclination is generally from 30 to 90°) rotating vessel (e.g. rotary plate or coating drum).

The temperatures which are necessary to bring about the removal of the bonding agent are below the highest calcination temperature for the catalyst, generally in the range from 200° C. to 600° C. The catalyst is preferably heated to from 240° C. to 500° C. and particularly preferably to temperatures in the range from 260° C. to 400° C. The time taken to remove the bonding agent can be a number of hours. The catalyst is generally heated for from 0.5 to 24 hours at the abovementioned temperature in order to remove the bonding agent. The time is preferably in the range from 1.5 to 8 hours and particularly preferably from 2 to 6 hours. Passing a gas over the catalyst can accelerate removal of the bonding agent. The gas is preferably air or nitrogen, particularly preferably air. Removal of the bonding agent can, for example, be carried out in a furnace through which a gas flows or in a suitable drying apparatus, for example a belt drier.

### Oxidative Dehydrogenation (Oxydehydrogenation, ODH)

An oxidative dehydrogenation of n-butenes to butadiene is carried out in a plurality of production cycles (i) by mixing a starting gas mixture comprising n-butenes with an oxygen-comprising gas and optionally additional inert gas or steam and bringing this mixture into contact with the catalyst arranged in a fixed catalyst bed at a temperature of from 220 to 490° C. in a fixed-bed reactor.

The reaction temperature of the oxydehydrogenation is generally controlled by means of a heat transfer medium which is present around the reaction tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 350 to 420° C.

Owing to the exothermic nature of the reactions which proceed, the temperature in particular sections of the interior of the reactor during the reaction can be higher than that of the heat transfer medium and a hot spot is formed. The position and height of the hot spot is determined by the reaction conditions, but can also be regulated by means of the dilution ratio of the catalyst bed or the flow of mixed gas.

The oxydehydrogenation can be carried out in all fixed-bed reactors known from the prior art, for example in tray furnaces, in a fixed-bed tube reactor or shell-and-tube reactor or in a plate heat exchanger reactor. A shell-and-tube reactor is preferred.

Furthermore, the catalyst bed which is installed in the reactor can consist of a single zone or of two or more zones. These zones can consist of pure catalyst or be diluted with a material which does not react with the starting gas or components of the product gas from the reaction. Furthermore, the catalyst zones can consist of all-active material or of supported coated catalysts.

As starting gas, it is possible to use pure n-butenes (1-butene and/or cis-/trans-2-butene) but also a gas mixture comprising butenes. Such a gas mixture can be obtained, for example, by nonoxidative dehydrogenation of n-butane. It is also possible to use a fraction which comprises n-butenes (1-butene and/or 2-butene) as main constituent and has been obtained from the C₄ fraction from cracking of naphtha by separating off butadiene and isobutene. Furthermore, gas mixtures which comprise pure 1-butene, cis-2-butene, trans-2-butene or mixtures thereof and have been obtained by dimerization of ethylene can also be used as starting gas. In addition, gas mixtures which comprise n-butenes and have been obtained by fluid catalytic cracking (FCC) can be used as starting gas.

In an embodiment of the process of the invention, the starting gas mixture comprising n-butenes is obtained by nonoxidative dehydrogenation of n-butane. Coupling of a nonoxidative catalytic dehydrogenation with the oxidative dehydrogenation of the n-butenes formed makes it possible to obtain a high yield of butadiene, based on n-butane used. The nonoxidative catalytic dehydrogenation of n-butane gives a gas mixture comprising butadiene, 1-butene, 2-butene and unreacted n-butane and also secondary constituents. Usual secondary constituents are hydrogen, water vapor, nitrogen, CO and CO₂, methane, ethane, ethene, propane and propene. The composition of the gas mixture leaving the first dehydrogenation zone can vary greatly as a function of the way in which the dehydrogenation is carried out. Thus, when the dehydrogenation is carried out with introduction of oxygen and additional hydrogen, the product gas mixture has a comparatively high content of water vapor and carbon oxides. In modes of operation without introduction of oxygen, the product gas mixture from the nonoxidative dehydrogenation has a comparatively high content of hydrogen.

The product gas stream from the nonoxidative dehydrogenation of n-butane typically comprises from 0.1 to 15% by volume of butadiene, from 1 to 15% by volume of 1-butene, from 1 to 25% by volume of 2-butene (cis/trans-2-butene), from 20 to 70% by volume of n-butane, from 1 to 70% by volume of water vapor, from 0 to 10% by volume of low-boiling hydrocarbons (methane, ethane, ethene, propane and propene), from 0.1 to 40% by volume of hydrogen, from 0 to 70% by volume of nitrogen and from 0 to 5% by volume of carbon oxides. The product gas stream from the nonoxidative dehydrogenation can be fed without further work-up to the oxidative dehydrogenation.

In a further embodiment of the invention, “raffinate II” is used. This can comprise the following components: from 25 to 70% by volume of 1-butene, from 20 to 60% by volume of 2-butene (cis/trans-2-butene), from 0 to 6% by volume of isobutene, 0.1-15% by volume of isobutane, from 3 to 30% by volume of n-butane and from 0.01 to 5% by volume of butadiene.

Furthermore, any impurities can be present in the starting gas for the oxydehydrogenation in a range in which the effect of the present invention is not inhibited. In the preparation of butadiene from n-butenes (1-butene and cis-/trans-2-butene), mention may be made of saturated and unsaturated, branched and unbranched hydrocarbons, e.g. methane, ethane, ethene, acetylene, propane, propene, propyne, n-butane, isobutane, isobutene, n-pentane and also dienes such as 1,2-butadiene, as impurities. The amounts of impurities are generally 70% or less, preferably 30% or less, more preferably 10% or less and particularly preferably 1% or less. The concentration of linear monoolefins having 4 or more carbon atoms (n-butenes and higher homologues) in the starting gas is not subject to any particular restrictions; it is generally 35.00-99.99% by volume, preferably 71.00-99.0% by volume and even more preferably 75.00-95.0% by volume.

To carry out the oxidative dehydrogenation at full conversion of butenes, it is necessary to use a gas mixture which has a molar oxygen: n-butene ratio of at least 0.5. Preference is given to working at an oxygen: n-butene ratio of from 0.55 to 10. To set this value, the starting gas can be mixed with oxygen or an oxygen-comprising gas, for example air, and optionally additional inert gas or steam. The oxygen-comprising gas mixture obtained is then fed to the oxydehydrogenation.

The gas comprising molecular oxygen is a gas which generally comprises more than 10% by volume, preferably more than 15% by volume and even more preferably more than 20% by volume, of molecular oxygen and is specifically preferably air. The upper limit to the content of molecular oxygen is generally 50% by volume or less, preferably 30% by volume or less and even more preferably 25% by volume or less. In addition, any inert gases can be present in the gas comprising molecular oxygen in a range in which the effect of the present invention is not inhibited. As possible inert gases, mention may be made of nitrogen, argon, neon, helium, CO, CO₂ and water. The amount of inert gases is generally 90% by volume or less, preferably 85% by volume or less and even more preferably 80% by volume or less, in the case of nitrogen. In the case of constituents other than nitrogen, it is generally 10% by volume or less, preferably 1% by volume or less. If this amount becomes too great, it becomes ever more difficult to supply the reaction with the necessary oxygen.

Furthermore, inert gases such as nitrogen and also water (as water vapor) can be comprised together with the mixed gas composed of starting gas and the gas comprising molecular oxygen. Nitrogen is present to set the oxygen concentration and to prevent formation of an explosive gas mixture; the same applies to steam. Furthermore, steam is present to control carbonization of the catalyst and to remove the heat of reaction. Preference is given to water (as water vapor) and nitrogen being mixed into the mixed gas and introduced into the reactor.

When water vapor is introduced into the reactor, preference is given to introducing a proportion of 0.2-5.0 (parts by volume), preferably 0.5-4 and even more preferably 0.8-2.5, based on the amount of the abovementioned starting gas introduced. When nitrogen gas is introduced into the reactor, preference is given to introducing a proportion of 0.1-8.0 (parts by volume), preferably 0.5-5.0 and even more preferably 0.8-3.0, based on the amount of the abovementioned starting gas introduced.

In general, the proportion of the starting gas comprising hydrocarbons in the mixed gas is 4.0% by volume or more, preferably 6.0% by volume or more and even more preferably 8.0% by volume or more. On the other hand, the upper limit is 20% by volume or less, preferably 16.0% by volume or less and even more preferably 13.0% by volume or less. To avoid the formation of explosive gas mixtures reliably, nitrogen gas is firstly introduced into the starting gas or into the gas comprising molecular oxygen before the mixed gas is obtained, the starting gas and the gas comprising molecular oxygen is mixed so as to obtain the mixed gas and this mixed gas is then preferably introduced.

The mixed gas fed into the fixed-bed reactor preferably has the following composition: from 2.5 to 7.5% by volume of 2-butene, from 2.5 to 6% by volume of 1-butene, with the total amount of n-butene (1- and 2-butenes) being in the range from 5.5 to 9% by volume, from 0 to 8% by volume of n-butane, from 0 to 3% by volume of isobutane, from 1 to 15% by volume of water vapor, from 0 to 0.5% by volume of low-boiling hydrocarbons (methane, ethane, ethene, propane and propene), from 9.5 to 13% by volume of oxygen, from 60 to 80% by volume of nitrogen, from 0 to 2% by volume of carbon oxides.

According to the invention, the oxygen content of the mixed gas fed into the fixed-bed reactor is selected so that the oxygen content of the product gas mixture leaving the fixed-bed reactor is still at least 5% by volume, preferably still at least 6% by volume.

The product gas stream leaving the oxidative dehydrogenation in the production step comprises butadiene and generally also unreacted n-butane and isobutane, 2-butene and water vapor. As secondary constituents, it generally comprises carbon monoxide, carbon dioxide, oxygen, nitrogen, methane, ethane, ethene, propane and propene, possibly hydrogen and also oxygen-comprising hydrocarbons, known as oxygenates. In general, it comprises only small proportions of 1-butene and isobutene.

For example, the product gas stream leaving the oxidative dehydrogenation can comprise from 4 to 8% by volume of butadiene, from 0 to 8% by volume of n-butane, from 0 to 3% by volume of isobutane, from 0.2 to 5% by volume of 2-butene, from 0 to 0.5% by volume of 1-butene, from 7 to 23% by volume of water vapor, from 0 to 0.5% by volume of low-boiling hydrocarbons (methane, ethane, ethene, propane and propene), from 0 to 10% by volume of hydrogen, from 5 to 8% by volume of oxygen, from 55 to 75% by volume of nitrogen, from 0 to 2% by volume of carbon oxides and from 0 to 1% by volume of oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

According to the invention, the oxygen content of the product gas mixture at the outlet from the fixed-bed reactor is at least 5% by volume, preferably at least 6% by volume, based on all constituents of the gas. In general, the oxygen content of the product gas mixture is not more than 8% by volume, preferably not more than 7% by volume.

During stable operation, the residence time in the reactor is not subject to any particular restrictions for the purposes of the present invention, but the lower limit is generally 0.3 s or more, preferably 0.7 s or more and even more preferably 1.0 s or more. The upper limit is 5.0 s or less, preferably 3.5 s or less and even more preferably 2.5 s or less. The ratio of throughput of mixed gas based on the amount of catalyst in the interior of the reactor is 500-8000 h⁻¹, preferably 800-4000 h⁻¹ and even more preferably 1200-3500 h⁻¹. The space velocity of butenes over the catalyst (expressed in g_{butenes}/(g_{catalyst}*hour) is generally 0.1-5.0 h⁻¹ in stable operation, preferably 0.2-3.0 h⁻¹ and even more preferably 0.25-1.0 h⁻¹. Volume and mass of the catalyst relate to the complete catalyst consisting of support and active composition.

### Regeneration of the Multimetal Oxide Catalyst

According to the invention, a regeneration step (ii) is carried out between each two production steps (i). The regeneration step (ii) is, according to the invention, carried out after a duration of the preceding product step of not more than 1000 hours, preferably not more than 670 hours, particularly preferably not more than 330 hours. The regeneration step (ii) is carried out by passing an oxygen-comprising regeneration gas mixture over the fixed catalyst bed at a temperature of from 200 to 450° C., as a result of which the carbon deposited on the multimetal oxide catalyst is burnt off.

The oxygen-comprising regeneration gas mixture used in the regeneration step (i) generally comprises an oxygen-comprising gas and additional inert gases, water vapor and/or hydrocarbons. An oxygen-comprising regeneration gas preferably comprises a proportion by volume of molecular oxygen of 0.1-21%, preferably 0.2-10% and even more preferably 0.25-5%, at the beginning of the regeneration.

A preferred oxygen-comprising gas present in the regeneration gas mixture is air. To produce the oxygen-comprising regeneration gas mixture, inert gases, water vapor and/or hydrocarbons can optionally be additionally mixed into the oxygen-comprising gas. Possible inert gases are nitrogen, argon, neon, helium, CO and CO₂. The amount of inert gases is generally 99% by volume or less, preferably 98% by volume or less and even more preferably 96% by volume or less, in the case of nitrogen. In the case of constituents other than nitrogen, it is generally 50% by volume or less, preferably 40% by volume or less. The proportion of inert gases can be decreased during the course of the regeneration. The amount of oxygen-comprising gas is selected in such a way that the proportion by volume of molecular oxygen in the regeneration gas mixture is 0.1-21%, preferably 0.2-10% and even more preferably 0.25-5%, at the beginning of the regeneration. The proportion of molecular oxygen can be increased during the course of the regeneration.

Furthermore, water vapor can also be comprised in the oxygen-comprising regeneration gas mixture. Nitrogen is present in order to set the oxygen concentration, and the same applies to water vapor. Water vapor can also be present to remove the heat of reaction and as mild oxidant for removing carbon-comprising deposits. Preference is given to introducing water (as water vapor) and nitrogen into the regeneration gas mixture and into the reactor. When water vapor is introduced into the reactor at the beginning of the regeneration, preference is given to introducing a proportion by volume of from 0 to 50%, preferably from 0 to 22% and even more preferably from 0.1 to 10%. The proportion of water vapor can be increased during the course of the regeneration. The amount of nitrogen is selected so that the proportion by volume of molecular nitrogen in the regeneration gas mixture at the beginning of the regeneration is from 20 to 99%, preferably from 50 to 98% and even more preferably from 70 to 96%. The proportion of nitrogen can be decreased during the course of the regeneration.

Furthermore, the regeneration gas mixture can comprise hydrocarbons. These can be additionally mixed in or introduced in place of the inert gases. The proportion by volume of hydrocarbons in the oxygen-comprising regeneration gas mixture is generally less than 50%, preferably less than 10% and even more preferably less than 5%. The hydrocarbons can comprise saturated and unsaturated, branched and unbranched hydrocarbons, e.g. methane, ethane, ethene, acetylene, propane, propene, propyne, n-butane, isobutane, n-butene, isobutene, n-pentane and also dienes such as 1,3-butadiene and 1,2-butadiene. In particular, they comprise hydrocarbons which have no reactivity in the presence of oxygen under the regeneration conditions in the presence of the catalyst.

During stable operation, the residence time of the regeneration gas mixture in the reactor during the regeneration is not subject to any particular restrictions, but the lower limit is generally 0.3 s or more, preferably 0.7 s or more and even more preferably 1.0 s or more. Very long residence times can be possible. The upper limit can be up to 10 000 seconds. It is generally 4000 s or less, preferably 400 s or less and even more preferably 40 s or less. The ratio of throughput of mixed gas based on the catalyst volume in the interior of the reactor is from 0.5 to 8000 h⁻¹, preferably from 10 to 4000 h⁻¹.

The reaction temperature of the regeneration is generally controlled by means of a heat transfer medium which is present around the reaction tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 350 to 420° C. All temperatures indicated above and below for the production steps (i) and regeneration steps (ii) relate to the temperature of the heat transfer medium at the inlet for the heat transfer medium on the reactor.

The temperature in the regeneration cycle (ii) is preferably in the same temperature range as in the production cycle (i). The temperature in the production cycle (i) is preferably above 350° C., particularly preferably above 360° C. and in particular above 365° C., and is preferably not more than 420° C. The temperatures mentioned relate to the temperature of the heat transfer medium at the inlet for the heat transfer medium on the reactor.

### Work-Up of the Product Gas Stream

The product gas stream leaving the oxidative dehydrogenation during the production step comprises butadiene and generally also unreacted n-butane and isobutane, 2-butene and water vapor. As secondary constituents, it generally comprises carbon monoxide, carbon dioxide, oxygen, nitrogen, methane, ethane, ethene, propane and propene, possibly hydrogen and also oxygen-comprising hydrocarbons, known as oxygenates. It generally also comprises small proportions of 1-butene and isobutene.

For example, the product gas stream leaving the oxidative dehydrogenation can comprise from 4 to 8% by volume of butadiene, from 0 to 8% by volume of n-butane, from 0 to 3% by volume of isobutane, from 0.2 to 5% by volume of 2-butene, from 0 to 0.5% by volume of 1-butene, from 7 to 23% by volume of water vapor, from 0 to 0.5% by volume of low-boiling hydrocarbons (methane, ethane, ethene, propane and propene), from 0 to 10% by volume of hydrogen, from 5 to 8% by volume of oxygen, from 55 to 75% by volume of nitrogen, from 0 to 2% by volume of carbon oxides and from 0 to 1% by volume of oxygenates. Oxygenates can be, for example, formaldehyde, furan, acetic acid, maleic anhydride, formic acid, methacrolein, methacrylic acid, crotonaldehyde, crotonic acid, propionic acid, acrylic acid, methyl vinyl ketone, styrene, benzaldehyde, benzoic acid, phthalic anhydride, fluorenone, anthraquinone and butyraldehyde.

The product gas stream at the reactor outlet is characterized by a temperature close to the temperature at the end of the catalyst bed. The product gas stream is then brought to a temperature of 150-400° C., preferably 160-300° C., particularly preferably 170-250° C. It is possible to insulate the line through which the product gas stream flows in order to keep the temperature in the desired range, but use of a heat exchanger is preferred. This heat exchange system can be of any type as long as the temperature of the product gas can be kept at the desired level by means of this system. Examples of heat exchangers are spiral heat exchangers, plate heat exchangers, double-tube heat exchangers, multitude heat exchangers, vessel-spiral heat exchangers, vessel-jacket heat exchangers, liquid-liquid contact heat exchangers, air heat exchangers, direct contact heat exchangers and finned tube heat exchangers. Since part of the high-boiling by-products comprised in the product gas can precipitate while the temperature of the product gas is brought to the desired temperature, the heat exchanger system should preferably have two or more heat exchangers. When two or more heat exchangers provided are arranged in parallel and divided cooling of the product gas in the heat exchangers is made possible, the amount of high-boiling by-products which deposit in the heat exchangers decreases and the operating life of the heat exchangers can thus be increased. As an alternative to the abovementioned method, the two or more heat exchangers provided can be arranged in parallel. The product gas is fed to one or more but not all heat exchangers and, after a particular period of operation, these heat exchangers are relieved by other heat exchangers. In this method, cooling can be continued, part of the heat of reaction can be recovered and, in parallel thereto, the high-boiling by-products deposited in one of the heat exchangers can be removed. As an organic solvent of the abovementioned type it is possible to use a solvent without restriction as long as it is able to dissolve the high-boiling by-products; as examples, it is possible to use an aromatic hydrocarbon solvent such as toluene, xylene, etc., or an alkaline aqueous solvent such as an aqueous solution of sodium hydroxide.

If the product gas stream comprises more than small traces of oxygen, a process step for removing residual oxygen from the product gas stream can be carried out. The residual oxygen can interfere because it can cause butadiene peroxide formation in downstream process steps and act as initiator for polymerization reactions. Unstable 1,3-butadiene can form dangerous butadiene peroxides in the presence of oxygen. The peroxides are viscous liquids. Their density is higher than that of butadiene. Since they also have only a low solubility in liquid 1,3-butadiene, they deposit at the bottom of storage containers. Despite their relatively low chemical reactivity, the peroxides are very unstable compounds which can decompose spontaneously at temperatures in the range from 85 to 110° C. A particular hazard is the high shock sensitivity of the peroxides which explore with the brisance of an explosive. The risk of polymer formation is, in particular, present in the isolation of butadiene by distillation and can there lead to deposits of polymers (formation of “popcorn”) in the columns. The oxygen removal is preferably carried out immediately after the oxidative dehydrogenation. In general, a catalytic combustion step in which the oxygen is reacted in the presence of a catalyst with hydrogen introduced in this step is carried out for this purpose. In this way, reduction of the oxygen content down to small traces is achieved.

The product gas from the O₂ removal step is then brought to an identical temperature level as has been described for the region downstream of the ODH reactor. Cooling of the compressed gas is carried out using heat exchangers which can, for example, be configured as shell-and-tube heat exchangers, spiral heat exchangers or plate heat exchangers. The heat removed here is preferably utilized for heat integration in the process.

A major part of the high-boiling secondary components and of the water can subsequently be separated off from the product gas stream by cooling. This separation is preferably carried out in a quench. This quench can consist of one or more stages. Preference is given to using a process in which the product gas is directly brought into contact with the cooling medium and cooled thereby. The cooling medium is not subject to any particular restrictions, but water or an alkaline aqueous solution is preferably used. It is also possible to use organic solvents, preferably aromatic hydrocarbons, particularly preferably toluene, o-xylene, m-xylene, p-xylene or mixtures thereof, as cooling medium. This gives a gas stream in which n-butane, 1-butene, 2-butenes, butadiene, possibly oxygen, hydrogen, water vapor, small amounts of methane, ethane, ethene, propane and propene, isobutane, carbon oxides and inert gases remain. Furthermore, traces of high-boiling components which have not been separated off quantitatively in the quench can remain in this product gas stream.

The product gas stream from the quench is subsequently compressed in a first compression stage and then cooled, as a result of which at least one condensate stream comprising water and possibly organic cooling medium is condensed out and a gas stream comprising n-butane, 1-butene, 2-butene, butadiene, possibly hydrogen, water vapor, small amounts of methane, ethane, ethene, propane and propene, isobutane, carbon oxides and inert gases, possibly oxygen and hydrogen, remains. The compression can be carried out in one or more stages. Overall, the gas is compressed from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multistage compression. The condensate stream generally comprises at least 80% by weight, preferably at least 90% by weight, of water and additionally comprises small amounts of low boilers, C4-hydrocarbons, oxygenates and carbon oxides.

Suitable compressors are, for example, turbocompressors, rotary piston compressors and reciprocating piston compressors. The compressors can, for example, be driven by an electric motor, an expander or a gas or steam turbine. Typical compression ratios (exit pressure: entry pressure) per compression stage are, depending on the construction type, in the range from 1.5 to 3.0. Cooling of the compressed gas is effected by means of heat exchangers, which can, for example, be configured as shell-and-tube heat exchangers, spiral heat exchangers or plate heat exchangers. Cooling water or heat transfer oils are used as coolants in the heat exchangers. In addition, preference is given to using air cooling employing blowers.

The stream comprising butadiene, butenes, butane, inert gases and possibly carbon oxides, oxygen, hydrogen and also low-boiling hydrocarbons (methane, ethane, ethene, propane, propene) and small amounts of oxygenates is fed as starting stream to the further treatment.

The removal of the low-boiling secondary constituents from the product gas stream can be effected by means of conventional separation processes such as distillation, rectification, membrane processes, absorption or adsorption.

To separate off any hydrogen comprised in the product gas stream, the product gas mixture can, optionally after cooling, for example in a heat exchanger, be passed over a generally tubular membrane which is permeable only to molecular hydrogen. The molecular hydrogen separated off in this way can, if necessary, be at least partly used in a hydrogenation or else passed to another use, for example be used for generating electric energy in fuel cells.

The carbon dioxide comprised in the product gas stream can be separated off by means of a CO₂ gas scrub. The carbon dioxide gas scrub can be preceded by a separate combustion stage in which the carbon monoxide is selectively oxidized to carbon dioxide.

In a preferred embodiment of the process, the incondensable or low-boiling gas constituents such as hydrogen, oxygen, carbon oxides, the low-boiling hydrocarbons (methane, ethane, ethene, propane, propene) and inert gas such as possibly nitrogen are separated off in an absorption/desorption cycle by means of a high-boiling absorption medium, giving a C₄ product gas stream which consists essentially of the C₄-hydrocarbons. In general, the C₄ product gas stream comprises at least 80% by volume, preferably at least 90% by volume, particularly preferably at least 95% by volume, of the C₄-hydrocarbons, essentially n-butane, 2-butene and butadiene.

For this purpose, the product gas stream is, after prior removal of water, brought into contact with an inert absorption medium in an absorption stage and the C₄-hydrocarbons are absorbed in the inert absorption medium, giving absorption medium loaded with C₄-hydrocarbons and an offgas comprising the other gas constituents. In a desorption stage, the C₄-hydrocarbons are liberated again from the absorption medium.

The absorption stage can be carried out in any suitable absorption column known to those skilled in the art. Absorption can be effected by simply passing the product gas stream through the absorption medium. However, it can also be carried out in columns or in rotary absorbers. The absorption can be carried out in concurrent, countercurrent or cross-current. The absorption is preferably carried out in countercurrent. Suitable absorption columns are, for example, tray columns having bubble cap trays, centrifugal trays and/or sieve trays, columns having structured packing, e.g. sheet metal packings having a specific surface area of from 100 to 1000 m²/m³, e.g. Mellapak® 250 Y, and columns packed with random packing elements. However, trickle towers and spray towers, graphite block absorbers, surface absorbers such as thick film absorbers and thin film absorbers and also rotary columns, plate scrubbers, crossed spray scrubbers and rotational scrubbers are also possible.

In an embodiment, the stream comprising butadiene, butene, butane and/or nitrogen and possibly oxygen, hydrogen and/or carbon dioxide is fed into the lower region of an absorption column. The stream comprising solvent and possibly water is introduced into the upper region of the absorption column.

Inert absorption media used in the absorption stage are generally high-boiling nonpolar solvents in which the C₄-hydrocarbon mixture to be separated off has a significantly higher solubility than the other gas constituents to be separated off. Suitable absorption media are comparatively nonpolar organic solvents, for example aliphatic C₈-C₁₈-alkanes, or aromatic hydrocarbons such as the middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight chain C₁-C₈-alkanols, and also heat transfer oils such as biphenyl and diphenyl ether, their chlorinated derivatives and also triarylalkenes. A suitable absorption medium is a mixture of biphenyl and diphenyl ether, preferably in the azeotropic composition, for example the commercially available Diphyl®. This solvent mixture frequently comprises dimethyl phthalate in an amount of from 0.1 to 25% by weight. Further preference is given to using organic solvents, preferably aromatic hydrocarbons, particularly preferably toluene, o-xylene, m-xylene, p-xylene or mixtures thereof, as absorption media.

Suitable absorption media are octanes, nonanes, decanes, undecanes, dodecanes, tridecanes, tetradecanes, pentadecanes, hexadecanes, heptadecanes and octadecanes or fractions which are obtained from refinery streams and comprise the abovementioned linear alkanes as main components.

In a preferred embodiment, an alkane mixture such as tetradecane (industrial C₁₄-C₁₇ fraction) is used as solvent for the absorption.

At the top of the absorption column, an offgas stream comprising essentially inert gas, carbon oxides, possibly butane, butenes such as 2-butenes and butadiene, possibly oxygen, hydrogen and low-boiling hydrocarbons (for example methane, ethane, ethene, propane, propene) and water vapor is taken off. Part of this stream can be fed to the ODH reactor or the O₂ removal reactor. In this way, the inlet stream into the ODH reactor can, for example, be set to the desired C₄-hydrocarbon content.

The solvent stream loaded with C₄-hydrocarbons is introduced into a desorption column. According to the invention, all column internals known to those skilled in the art are suitable for this purpose. In one process variant, the desorption step is carried out by depressurizing and/or heating the loaded solvent. A preferred process variant is the addition of stripping steam and/or the introduction of fresh steam into the bottom of the desorber. The solvent depleted in C₄-hydrocarbons can be fed as mixture together with the condensed steam (water) to a phase separation, so that the water is separated off from the solvent. All apparatuses known to those skilled in the art are suitable for this purpose. In addition, it is possible to utilize the water separated off from the solvent for generating the stripping steam.

Preference is given to using from 70 to 100% by weight of solvent and from 0 to 30% by weight of water, particularly preferably from 80 to 100% by weight of solvent and from 0 to 20% by weight of water, in particular from 85 to 95% by weight of solvent and from 5 to 15% by weight of water. The absorption medium which has been regenerated in the desorption stage is recirculated to the absorption stage.

The separation is generally not quite complete, so that, depending on the type of separation, small amounts or even only traces of the further gas constituents, in particular high-boiling hydrocarbons, can be present in the C₄ product gas stream. The reduction in the volume flow also brought about by the separation reduces the load on the subsequent process steps.

The C₄ product gas stream consisting essentially of n-butane, butenes such as 2-butenes and butadiene generally comprises from 20 to 80% by volume of butadiene, from 20 to 80% by volume of n-butane, from 0 to 10% by volume of 1-butene and from 0 to 50% by volume of 2-butenes, where the total amount is 100% by volume. Furthermore, it can comprise small amounts of isobutane.

The C₄ product gas stream can subsequently be separated by means of extractive distillation into a stream consisting essentially of n-butane and 2-butene and a stream comprising butadiene. The stream consisting essentially of n-butane and 2-butene can be recirculated in its entirety or in part to the C₄ feed of the ODH reactor. Since the butene isomers in this recycle stream consist essentially of 2-butenes and these 2-butenes are generally oxidatively dehydrogenated more slowly to butadiene than is 1-butene, this recycled stream can go through a catalytic isomerization process before being fed into the ODH reactor. In this catalytic process, the isomer distribution can be set to the isomer distribution present at thermodynamic equilibrium.

The extractive distillation can, for example, be carried out as described in “Erdöl und Kohle-Erdgas-Petrochemie”, volume 34 (8), pages 343 to 346 or “Ullmanns Enzyklopädie der Technischen Chemie”, volume 9, 4 edition 1975, pages 1 to 18. For this purpose, the C₄ product gas stream is brought into contact with an extractant, preferably an N-methylpyrrolidone (NMP)/water mixture, in an extraction zone. The extraction zone is generally configured in the form of a scrubbing column which comprises trays, random packing elements or ordered packing as internals. These generally have from 30 to 70 theoretical plates in order to achieve a sufficiently good separation action. The scrubbing column preferably has a backwashing zone in the top of the column. This backwashing zone serves to recover the extractant comprised in the gas phase by means of a liquid hydrocarbon runback, for which purpose the overhead fraction is condensed beforehand. The mass ratio of extractant to C₄ product gas stream in the feed to the extraction zone is generally from 10:1 to 20:1. The extractive distillation is preferably carried out at a temperature at the bottom in the range from 100 to 250° C., in particular at a temperature in the range from 110 to 210° C., a temperature at the top in the range from 10 to 100° C., in particular in the range from 20 to 70° C., and a pressure in the range from 1 to 15 bar, in particular in the range from 3 to 8 bar. The extractive distillation column preferably has from 5 to 70 theoretical plates.

Suitable extractants are butyrolactone, nitriles such as acetonitrile, propionitrile, methoxypro-pionitrile, ketones such as acetone, furfural, N-alkyl-substituted lower aliphatic acid amides such as dimethylformamide, diethylformamide, dimethylacetamide, diethylacetamide, N-formylmorpholine, N-alkyl-substituted cyclic acid amides (lactams) such as N-alkylpyrrolidones, in particular N-methylpyrrolidone (NMP). In general, alkyl-substituted lower aliphatic acid amides or N-alkyl-substituted cyclic acid amides are used. Dimethylformamide, acetonitrile, furfural and in particular NMP are particularly advantageous.

However, it is also possible to use mixtures of these extractants with one another, e.g. of NMP and acetonitrile, mixtures of these extractants with cosolvents and/or tert-butyl ethers, e.g. methyl tert-butyl ether, ethyl tert-butyl ether, propyl tert-butyl ether, n-butyl or isobutyl tert-butyl ether. NMP, preferably in aqueous solution, more preferably with from 0 to 20% by weight of water, particularly preferably with from 7 to 10% by weight of water, in particular with 8.3% by weight of water, is particularly useful.

The overhead product stream from the extractive distillation column comprises essentially butane and butenes and small amounts of butadiene and is taken off in gaseous or liquid form. In general, the stream consisting essentially of n-butane and 2-butene comprises from 50 to 100% by volume of n-butane, from 0 to 50% by volume of 2-butene and from 0 to 3% by volume of further constituents such as isobutane, isobutene, propane, propene and C s-hydrocarbons.

A stream comprising the extractant, water, butadiene and small proportions of butenes and butane is obtained at the bottom of the extractive distillation column and is fed to a distillation column. In the latter, butadiene is obtained at the top or as a side offtake stream. A stream comprising extractant and water is obtained at the bottom of the distillation column; the composition of the stream comprising extractant and water corresponds to the composition as is introduced into the extraction. The stream comprising extractant and water is preferably recirculated to the extractive distillation.

The extraction solution is transferred to a desorption zone in which the butadiene is desorbed from the extraction solution. The desorption zone can, for example, be configured in the form of a scrubbing column which has from 2 to 30, preferably from 5 to 20, theoretical plates and optionally a backwashing zone having, for example, 4 theoretical plates. This backwashing zone serves to recover the extractant comprised in the gas phase by means of a liquid hydrocarbon runback, for which purpose the overhead fraction is condensed beforehand. Ordered packing, trays or random packing elements are provided as internals. The distillation is preferably carried out at a temperature at the bottom in the range from 100 to 300° C., in particular in the range from 150 to 200° C., and a temperature at the top in the range from 0 to 70° C., in particular in the range from 10 to 50° C. The pressure in the distillation column is preferably in the range from 1 to 10 bar. In general, a lower pressure and/or a higher temperature than in the extraction zone prevail(s) in the desorption zone.

The desired product stream obtained at the top of the column generally comprises from 90 to 100% by volume of butadiene, from 0 to 10% by volume of 2-butene and from 0 to 10% by volume of n-butane and isobutane. To purify the butadiene further, a further distillation according to the prior art can be carried out.

The invention is illustrated in more detail by the following examples.

The parameters conversion (X) and selectivity (S) calculated in the examples were determined as follows:

$$X = \frac{mol ( {butene}_{in} ) - mol ( {butene}_{out} )}{mol ( {butene}_{in} )}$$

$$S = \frac{mol ( {butadiene}_{out} ) - mol ( {butadiene}_{in} )}{mol ( {butene}_{in} ) - mol ( {butene}_{out} )}$$

where mol(XXXᵢₙ) is the molar amount of the component XXX at the reactor inlet, mol(XXXₒᵤₜ) is the molar amount of the component XXX at the reactor outlet and butenes is the sum of 1-butene, cis-2-butene, trans-2-butene and isobutene.

### EXAMPLES

### Catalyst Production

### Example 1

2 Solutions A and B were produced.

### Solution A.

3200 g of water were placed in a 10 l stainless steel pot. While stirring by means of an anchor stirrer, 5.2 g of a KOH solution (32% by weight of KOH) were added to the initially charged water. The solution was heated to 60° C. 1066 g of an ammonium heptamolybdate solution ((NH₄)₆Mo₇O₂₄*4 H₂0, 54% by weight of Mo) were then added a little at a time over a period of 10 minutes. The suspension obtained was stirred for another 10 minutes.

### Solution B:

1771 g of a cobalt (II) nitrate solution (12.3% by weight of Co) were placed in a 5 l stainless steel pot and heated to 60° C. while stirring (anchor stirrer). 645 g of an iron(III) nitrate solution (13.7% by weight of Fe) were then added a little at a time over a period of 10 minutes while maintaining the temperature. The solution formed was stirred for another 10 minutes. 619 g of a bismuth nitrate solution (10.7% by weight of Bi) were then added while maintaining the temperature. After stirring for a further 10 minutes, 109 g of chromium(III) nitrate was added a little at a time as a solid and the dark red solution formed was stirred for another 10 minutes.

While maintaining the temperature of 60° C., the solution B was pumped into the solution A over a period of 15 minutes by means of a peristaltic pump. During the addition and thereafter, the mixture was stirred by means of a high-speed mixer (Ultra-Turrax). After the addition was complete, the mixture was stirred for another 5 minutes. 93.8 g of an SiO₂ suspension (Ludox; SiO₂ about 49%, from Grace) were then added and the mixture was stirred for a further 5 minutes.

The suspension obtained was spray dried over a period of 1.5 hours in a spray drier from NIRO (spray head No. FOA1, speed of rotation 25 000 rpm). During this operation, the temperature of the reservoir was maintained at 60° C. The gas inlet temperature of the spray drier was 300° C., and the gas outlet temperature was 110° C. The powder obtained had a particle size (d₅₀) of less than 40 μm.

The powder obtained was mixed with 1% by weight of graphite, compacted twice under a pressing pressure of 9 bar and comminuted through a sieve having a mesh opening of 0.8 mm. The crushed material was again mixed with 2% by weight of graphite and the mixture was pressed on a Kilian S100 tableting press to give 5×3×2 mm rings (external diameter×length×internal diameter).

The catalyst precursor obtained was calcined batchwise (500 g) in a convection furnace from Heraeus, DE (type K, 750/2 S, internal volume 55 I). The following program was used for this purpose:
heating over a period of 72 minutes to 130° C., hold for 72 minutes
heating over a period of 36 minutes to 190° C., hold for 72 minutes
heating over a period of 36 minutes to 220° C., hold for 72 minutes
heating over a period of 36 minutes to 265° C., hold for 72 minutes
heating over a period of 93 minutes to 380° C., hold for 187 minutes
heating over a period of 93 minutes to 430° C., hold for 187 minutes
heating over a period of 93 minutes to 490° C., hold for 467 minutes

After calcination, the catalyst having the calculated stoichiometry Mo₁₂Co₇Fe₃Bi_{0.6}K_{0.08}Cr_{0.5} Si_{1.6}Oₓ was obtained.

The calcined rings were milled to a powder.

### Example 2

Support bodies (steatite rings having dimensions of 5×3×2 mm (external diameter×height×internal diameter) were coated with the powder from example 1. For this purpose, 1054 g of the support were placed in a coating drum (2 l internal volume, angle of inclination of the central drum axis relative to the horizontal=30°). The drum was set into rotation (25 rpm). About 60 ml of liquid binder (mixture of glycerol:water 1:3) were sprayed (spraying air 500 standard l/h) onto the support over a period of about 30 minutes by means of an atomizer nozzle operated using compressed air. The nozzle was installed in such a way that the spray cone wetted the support bodies conveyed in the drum in the upper half of the rolling-down section. 191 g of the finely pulverulent precursor composition of the milled catalyst from example 1 were introduced by means of a powder screw into the drum, with the point of introduction of the powder being located within the rolling-down section but above the spray cone. The powder was introduced in such a way that uniform distribution of the powder on the surface was obtained. After coating was complete, the resulting coated catalyst composed of precursor composition and the support body was dried at 300° C. in a drying oven for 4 hours.

### Example 3

Support bodies (steatite rings having dimensions of 7×3×4 mm (external diameter×height×internal diameter) were coated with the powder from example 1. For this purpose, 900 g each time of the support were placed three times in a coating drum (2 l internal volume, angle of inclination of the central drum axis relative to the horizontal=30°). The drum was set in rotation (36 rpm). About 70 ml of liquid binder (mixture of glycerol:water 1:3) were sprayed (spraying air 200 standard l/h) onto the support over a period of about 45 minutes by means of an atomizer nozzle operated using compressed air. The nozzle was installed in such a way that the spray cone wetted the support bodies conveyed in the drum in the upper half of the rolling-down section. 230 g in each case of the finely pulverulent precursor composition of the milled catalyst from example 1 were introduced into the drum by means of a powder screw, with the point of introduction of the powder being located within the rolling-down section but below the spray cone. The powder was introduced in such a way that uniform distribution of the powder on the surface was obtained. After coating was complete, the resulting coated catalyst composed of precursor composition and the support body was dried at 300° C. in a drying oven for 4 hours.

### Dehydrogenation Experiments

Dehydrogenation experiments were carried out in a screening reactor. The screening reactor was a salt bath reactor having a length of 120 cm and an internal diameter of 14.9 mm and a thermocouple sheath having an external diameter of 3.17 mm. A multiple temperature sensor having 7 measurement points was located in the thermocouple sheath. The lowermost four measurement points had a spacing of 10 cm and the uppermost four measuring points had a spacing of 5 cm. Butane and raffinate II or 1-butene were introduced in liquid form at about bar through a coriolis flow meter, mixed in a static mixer and subsequently depressurized and vaporized in a heated vaporization section. This gas was then mixed with nitrogen and introduced into a preheater having a steatite bed. Water was introduced in liquid form and vaporized in a stream of air in a vaporizer coil. The air/water vapor mixture was combined with the N2/raffinate II/butane mixture in the lower region of the preheater. The completely mixed feed gas was then fed into the reactor, with an analysis stream for on-line GC measurement being able to be taken off. An analysis stream is likewise taken off from the product gas leaving the reactor and can be analyzed by on-line GC measurement or by means of an IR analyzer to determine the proportion by volume of CO and CO₂. A pressure regulating valve is located downstream of the branch for the analysis line and sets the pressure level in the reactor.

### Example 4

A 6 cm long after-bed consisting of 16 g of steatite balls having a diameter of 3.5-4.5 mm was placed on the catalyst seat at the lower end of the screening reactor. 44 g of the catalyst from example 2 were then thoroughly mixed with 88 g of steatite rings having the same geometry and introduced into the reactor (146 ml bed volume, 88 cm bed height). The catalyst bed was followed by a 7 cm long preliminary bed consisting of 16 g of steatite balls having a diameter of from 3.5 to 4.5 mm.

The reactor was operated using 200 standard l/h of a reaction gas having the composition 8% by volume of butenes, 2% by volume of butane, from 7.3 to 12.2% by volume of oxygen, 12% by volume of water and nitrogen as main residual constituent at a salt bath temperature of 380° C. The product gases were analyzed by means of GC. The conversion and selectivity data, time on stream of the experiment and amount of carbon deposited are shown in table 1.

A mixture of 2.5% by volume of oxygen, 95% by volume of nitrogen and 2.5% by volume of water vapor was then passed over the catalyst for 20 hours while heating to 400° C. The carbon oxides formed were recorded by means of an IR measurement instrument. The amount of carbon burnt off is shown in table 1.

| | | | O₂ | O₂ | | | | | |
|---|---|---|---|---|---|---|---|---|---|
| Time on | | | Concentration | Concentration | | | | Amount of | Amount of |
| stream of the | | Flow rate | at the reactor | at the reactor | | | Amount of | butadiene | carbon [ppm |
| experiments | Temperature | [standard | inlet [% by | outlet [% by | Conversion | Selectivity | carbon | produced | based on |
| [days] | [° C.] | l/h] | volume] | volume] | of butenes | to butadiene | deposited [g] | [g] | butadiene] |
| 1 | 380 | 200 | 8.47 | 3.23 | 87.63 | 83.57 | 32.9 | 710 | 46.4 |
| 1 | 380 | 200 | 8.50 | 3.55 | 84.82 | 85.27 | 33.2 | 701 | 47.3 |
| 1 | 380 | 200 | 8.42 | 3.37 | 86.49 | 84.17 | 35.4 | 705 | 50.2 |
| 3 | 380 | 200 | 8.49 | 3.53 | 84.72 | 85.03 | 237.1 | 2094 | 113.2 |
| 5 | 380 | 200 | 8.31 | 3.44 | 83.61 | 78.00 | 1962 | 3160 | 620.9 |
| Average | | | | 3.42 | 85.5 | 83.2 | | | |
| 1 | 380 | 200 | 10.15 | 4.61 | 89.9 | 82.3 | 62.1 | 717 | 86.6 |
| 6 | 380 | 200 | 10.28 | 5.04 | 87.4 | 83.4 | 338 | 4241 | 79.6 |
| 3 | 380 | 200 | 10.332 | 5.002 | 85.6 | 85.7 | 57.6 | 2124 | 27.1 |
| Average | | | | 4.88 | 88.7 | 82.5 | | | |
| 2 | 380 | 200 | 7.30 | 2.26 | 85.2 | 81.9 | 543 | 1953 | 401.8 |
| 1.2 | 380 | 200 | 7.68 | 2.59 | 82.2 | 83.5 | 112 | 795 | 141.4 |
| Average | | | | 2.42 | 83.7 | 82.7 | | | |
| 17 | 380 | 200 | 12.20 | 6.30 | 86.8 | 90.4 | 27 | 12866 | 21 |

| | | | |
|---|---|---|---|
| Average | 6.30 | 86.8 | 90.4 |

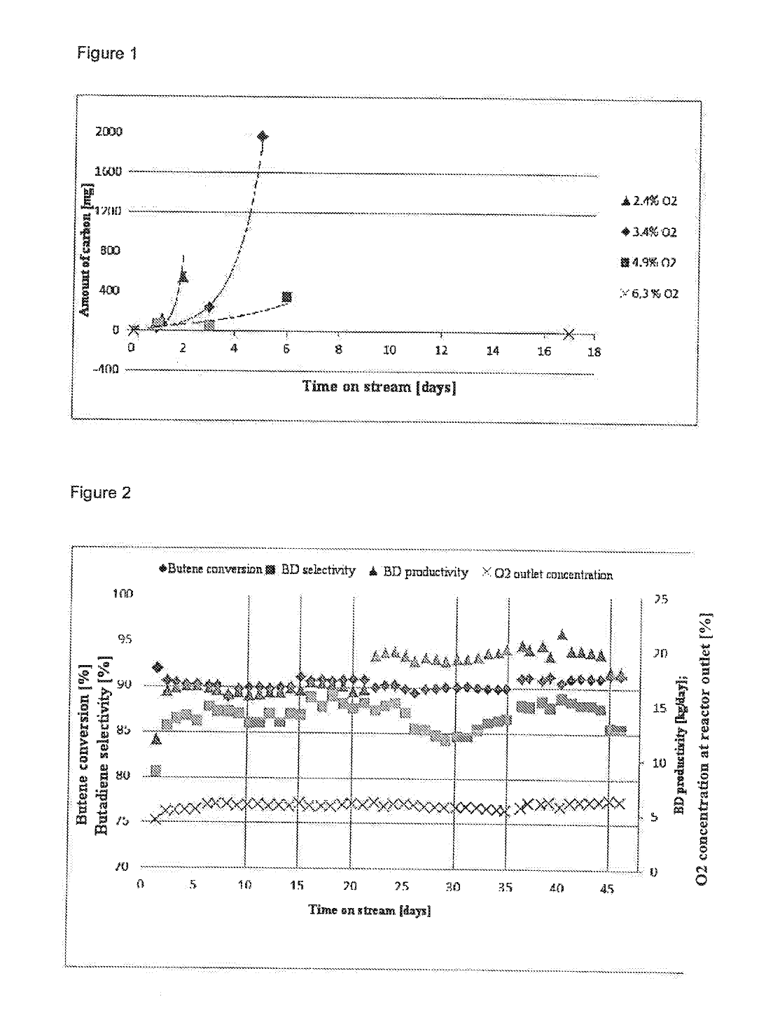

FIG. 1 shows the amount of carbon burnt off as a function of the reaction time for various oxygen concentrations at the reactor outlet: 2.4% by volume (▴), 3.4% by volume (♦), 4.9% by volume (▪), 6.3% by volume (x). The amount of carbon deposits increases exponentially with time for oxygen concentrations from 2.4 to 4.9% by volume. In the case of an oxygen concentration of 6.3% by volume, the amount of carbonaceous material is lower than the inaccuracy of the measurement.

### Example 5

Dehydrogenation experiments were carried out in a miniplant reactor. The miniplant reactor was a salt bath reactor having a length of 500 cm and an internal diameter of 29.7 mm and a thermocouple sheath having an external diameter of 6 mm. A 10 cm long after-bed consisting of 60 g of steatite rings having the geometry 7 mm×7 mm×4 mm (extemal diameter×length×internal diameter) was located on a catalyst seat. This was followed by 2756 g of an undiluted coated catalyst from example 3 (active composition content 20.2% by weight; bed height 384 cm, 2552 ml bed volume in the reactor) in the form of hollow cylinders having the dimensions 7 mm×3 mm×4 mm (external diameter×length×internal diameter). The catalyst bed was followed by an 85 cm long preliminary bed consisting of 494 g of steatite rings having the geometry 7 mm×7 mm×4 mm (external diameter×length×internal diameter).

The reaction tube was heated along its entire length by means of a flowing salt bath. A mixture of a total of 8% by volume of 1-, cis-2- and trans-2-butenes, 2% by volume of butanes (n-butane and isobutane), 12% by volume of oxygen, 12% by volume of water and 66% by volume of nitrogen was used as reaction starting gas mixture. The throughput through the reaction tube was 4500 standard l/h of total gas over the first 21 days and then 5500 standard l/h of total gas. The temperature of the salt after start up to and including day 21 was 372° C., and on the subsequent days was 377° C.

FIG. 2 shows the butene conversion (♦) in %, the butadiene selectivity (▪) in %, the butadiene productivity (▴) in kg/day and the oxygen concentration (x) in the product gas at the reactor outlet in % by volume as a function of the reaction time in days. After 46 days, the experiment was stopped and the amount of carbonaceous material deposited on the catalyst was determined. 102 g of carbon were burnt off, which corresponds to an amount of 120 ppm by weight based on butadiene produced. The catalyst was then removed from the reactor. Many places where spalling had occurred were observed on the catalyst. A statistically significant sample of rings was taken and the remaining active composition was washed in an ammoniacal ultrasonic bath. The uncoated rings obtained were dried and weighed again. While the active composition content of the catalyst from example 3 was still 20.2% by weight, the active composition content of the sample removed from the reactor was 15.8% by weight.

1.-9. (canceled)

10. A process for the oxidative dehydrogenation of n-butenes to butadiene, which comprises two or more production steps (i) and at least one regeneration step (ii) and in which
(i) mixing a starting gas mixture comprising n-butenes with an oxygen-comprising gas in a production step and the mixed gas is brought into contact with a multimetal oxide catalyst which comprises at least molybdenum and a further metal and is arranged in a fixed catalyst bed at a temperature of from 220 to 490° C. in a fixed-bed reactor, with a product gas mixture comprising at least butadiene, oxygen and water vapor being obtained at the outlet of the fixed-bed reactor,
and
(ii) regenerating the multimetal oxide catalyst in a regeneration step by passing an oxygen-comprising regeneration gas mixture over the fixed catalyst bed at a temperature of from 200 to 450° C. and burning off the carbon deposited on the catalyst,
with a regeneration step (ii) being carried out between two production steps (i),
wherein the oxygen content in the product gas mixture at the outlet of the fixed-bed reactor is at least 5% by volume and the duration of a production step (i) is not more than 1000 hours.

11. The process according to claim 10, wherein the duration of a production step (i) is less than 670 hours.

12. The process according to claim 10, wherein the oxygen content of the product gas mixture at the outlet of the fixed-bed reactor is at least 6% by volume.

13. The process according to claim 10, wherein the oxygen-comprising regeneration gas mixture comprises from 0.5 to 22% by volume of oxygen.

14. The process according to claim 10, wherein the oxygen-comprising regeneration gas mixture comprises from 0 to 30% by volume of water vapor.

15. The process according to claim 10, wherein the temperature in the production steps (i) is from 330 to 420° C.

16. The process according to claim 10, wherein the temperature in the regeneration steps (ii) is from 0 to 10° C. above or below the temperature in the production steps (i).

17. The process according to claim 10, wherein the multimetal oxide comprising molybdenum and at least one further metal has the general formula (I)
Mo₁₂BiₐFe_{b}Co_{c}Ni_{d}CrₑX¹_{f}X²_{g}Oₓ (I)
the variables having the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7;
b=0 to 5;
c=0 to 10;
d=0 to 10;
e=0 to 5;
f=0 to 24;
g=0 to 2; and
x=a number determined by the valence and abundance of the elements other than oxygen in (I).

18. The process according to claim 10, wherein the multimetal oxide comprising molybdenum and at least one further metal has the general formula (I)
Mo₁₂BiₐFe_{b}Co_{c}Ni_{d}CrₑX¹_{f}X²_{g}Oₓ (I)
the variables having the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.3 to 1.5;
b=2 to 4;
c=3 to 10;
d=0 to 10;
e=0 to 2;
f=0 to 2;
g=0.01 to 1; and
x=a number determined by the valence and abundance of the elements other than oxygen in (I).

19. The process according to claim 10, wherein the regeneration step (ii) is carried out when the conversion of the n-butenes has dropped by not more than 2% in the last 200 hours of the preceding production step (i).

20. The process according to claim 18, wherein the duration of a production step (i) is less than 670 hours and
the oxygen content of the product gas mixture at the outlet of the fixed-bed reactor is at least 6% by volume and
the oxygen-comprising regeneration gas mixture comprises from 0.5 to 22% by volume of oxygen.

21. The process according to claim 20, wherein the oxygen-comprising regeneration gas mixture comprises from 0 to 30% by volume of water vapor.

22. The process according to claim 21, wherein the temperature in the production steps (i) is from 330 to 420° C.

23. The process according to claim 22, wherein the temperature in the regeneration steps (ii) is from 0 to 10° C. above or below the temperature in the production steps (i).
